(19) United States
(12) Patent Application Publication
(10) Pub. No.: US 2004/0033613 A1
(43) Pub. Date: Feb. 19, 2004

(54) Saliva-based protein profiling

(76) Inventors: Michael G. Zwick, Monticello, WI (US); Charles F. Streckfus, Madison, MS (US); Lanora G. Bigler, Clinton, MS (US)

Correspondence Address: Richard S. Myers, Jr., Stites & Harbison PLLC, 424 Church Street, Suite 1800, Nashville, TN, 37219-2376, US

(21) Appl. No.: 10/448,624

(22) Filed: May 30, 2003

Related U.S. Application Data
(60) Provisional application No. 60/384,316, filed on May 30, 2002.

(51) Int. Cl. G01N 33/00

(52) U.S. Cl. 436/86

(57) ABSTRACT

The invention provides methods for profiling proteins present in saliva, and determination of differentials in protein profiles for analysis of gene function, differential gene expression, protein discovery, cellular and clinical diagnostics and drug screening.

### PRIORITY INFORMATION

This application claims priority to U.S. Application No. 60/384,316, filed May 30, 2002, now abandoned, the contents of which are incorporated herein by reference.

### FIELD OF THE INVENTION

The present invention relates to methods and systems of profiling protein content of biological samples such as saliva, for analysis of gene function, differential gene expression, protein discovery, cellular and clinical diagnostics and drug screening.

### BACKGROUND OF THE INVENTION

Cell function, both normal and pathologic, depends, in part, on the genes expressed by the cell (i.e., gene function). Gene expression has both qualitative and quantitative aspects. That is, cells may differ both in terms of the particular genes expressed and in terms of relative level of expression of the same gene. Differential gene expression can be manifested, for example, by differences in the expression of proteins encoded by the gene, or in post-translational modifications of expressed proteins. For example, proteins can be decorated with carbohydrates or phosphate groups, or they can be processed through peptide cleavage. Thus, at the biochemical level, a cell represents a complex mixture of organic biomolecules.

One goal of functional genomics (“proteomics”) is the identification and characterization of proteins that are differentially expressed between cell types. By comparing expression one can identify molecules that may be responsible for a particular pathologic activity of a cell. For example, identifying a protein that is expressed in cancer cells but not in normal cells is useful for diagnosis and, ultimately, for drug discovery and treatment of the pathology. Upon completion of the Human Genome Project, all the human genes will have been cloned, sequenced and organized in databases. However, it has become evident that knowledge of gene sequences or the quantity of gene expression is not sufficient to predict the biological nature and function of a protein. This can be particularly important when post-translational modifications of a protein have a significant impact on the function of the protein. For example, in cancer research, it has been found that post-translational modification can specifically contribute to the disease.

The ability to characterize the expression from multiple genes simultaneously, i.e., parallel analysis, has been recognized as a powerful approach to identifying factors involved in physiology, development, and disease. Such parallel analysis of proteins has applications is diagnosis of disease, identification of therapeutic markers and targets and in assessing response to pharmaceuticals.

A number of proteomics tools have been developed for the analysis and comparison of complex mixtures of proteins. Analysis of such mixtures can be referred to as “protein profiling”. Two-dimensional (2-D) protein gel electrophoresis is a widely used tool for display of protein expression profiles. To facilitate rapid analysis of expressed proteins in small samples such as microdissected tissue or small biopsies, methods using mass spectrometry (MS) for resolution have been developed. In particular, surface-enhanced laser desorption/ionization (SELDI) time-of-flight (TOF) analysis has been shown to be effective at determining changes in protein patterns from such samples.

However, even with these powerful tools for resolving protein profiles, acquisition and preparation of sample materials for such studies is burdensome. Collection of tissue samples from blood or biopsies for protein analysis is invasive to the test subject or patient. Consequently, the samples are more difficult to obtain for large-scale studies for biomarker discovery. Samples from healthy subjects who are not undergoing testing for other purposes may be especially difficult to obtain for such studies.

Further, such tissue samples generally require time-consuming preparation to purify components before analysis. Classical methods of sample purification, such as liquid chromatography (ion exchange, size exclusion, affinity, and reverse phase chromatography), membrane dialysis, centrifugation, immunoprecipitation, and electrophoresis, typically demand a large quantity of starting sample. Even when such quantities of sample are available, minor components tend to become lost in these purification processes, which suffer from analyte loss due to non-specific binding and dilution effects. The methods are also often quite labor intensive. Furthermore, complex biological materials, such as blood, sera, plasma, lymph, interstitial fluid, urine, exudates, whole cells, cell lysates and cellular secretion products, typically contain hundreds of biological molecules, plus organic and inorganic salts, which can complicate direct mass spectrometry analysis. Thus, significant sample preparation and purification steps are typically necessary prior to investigation.

Finally, in an economy-conscious environment in which cost-effective medicine is an overriding concern, there is a need for convenient, efficient methods for protein profiling for rapidly diagnosis and evaluation of responses to therapy.

There remains a need for methods of protein profiling using samples that can be easily obtained. The present invention meets this and other needs.

### SUMMARY OF THE INVENTION

In one aspect, the present invention provides a method for analyzing at least one test protein in a saliva sample. In preferred embodiments, the protein profile of a test sample of saliva is generated. In particularly preferred embodiments, the protein profile of a test sample is compared to the protein profile of a control sample.

In some embodiments, the invention provides a method for detecting proteins that are differentially present in a first and a second saliva sample comprising the steps of providing a first saliva sample and a second saliva sample, determining a first protein profile for said first saliva sample, determining a second protein profile for said second saliva sample, and comparing said first protein profile and said second protein profile to detect proteins that are differentially present in the first and second saliva samples. In preferred embodiments, the first and said second saliva samples are from a test subject and a control subject, respectively. In particularly preferred embodiments, the test subject is a person having a particular medical condition and the control subject is a person with a negative diagnosis for said particular medical condition.

In some embodiments, a plurality of saliva samples are collected from a single test subject. In some embodiments, the plurality of saliva samples are collected at different times. In some preferred embodiments, the protein profiles of the samples collected at different times are compared to assess the effect of a treatment provided at a time between the collections of the samples. In other preferred embodiments, the protein profiles of samples collected at different times are compared to detect the development of a condition associated with a particular biomarker. In yet other preferred embodiments, the protein profiles of samples collected at different times are compared to assess the progress of a condition associated with a particular biomarker.

The present invention provides methods of identifying saliva biomarkers for a particular phenotype, comprising providing a first saliva sample from a test subject having a phenotype of interest, providing a second saliva sample from a control subject not having said phenotype, determining a first protein profile for said first saliva sample, determining a second protein profile for said second saliva sample, and comparing said first protein profile and said second protein profile to detect proteins that are differentially present in the first and second saliva sample.

In some embodiments, protein profiles for samples are created by 2-D protein gel electrophoresis. In other embodiments, protein profiles are created by mass spectrometry. In preferred embodiments, protein profiles from saliva samples are created by SELDI-TOF mass spectrometry. In particularly preferred embodiments, SELDI-TOF MS-created protein profiles of saliva from a test population are compared with SELDI-OF MS-created protein profiles from a control population, to identify proteins that are expressed differentially.

### BRIEF DESCRIPTION OF THE DRAWINGS

FIG. 1 shows a diagram of the SELDI protein analysis process.

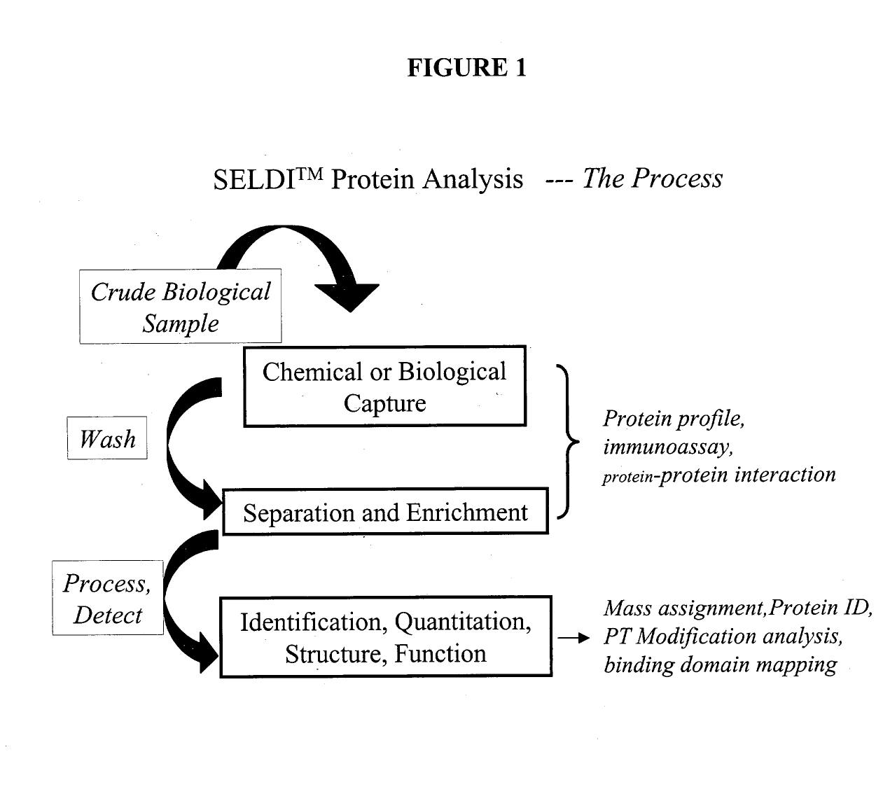

FIG. 2 shows data as it appears in a Spectral View (top) and a Gel View (bottom).

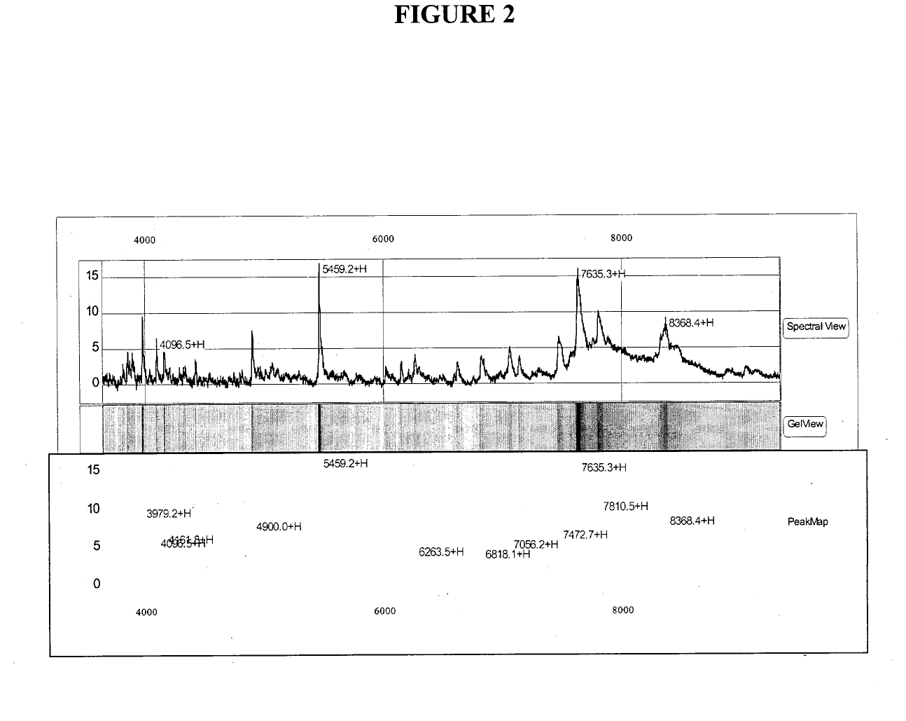

FIG. 3 shows an example of clustering analysis of the Ciphergen SELDI software, which allows grouping of similar masses across multiple spectra. The spectra here are generated from LCM-procured cancer cells from 8 patients (4 class I and 4 class II). Potential differences (patterns) are visualized by generating a scatter plot of the intensity vs. cluster.

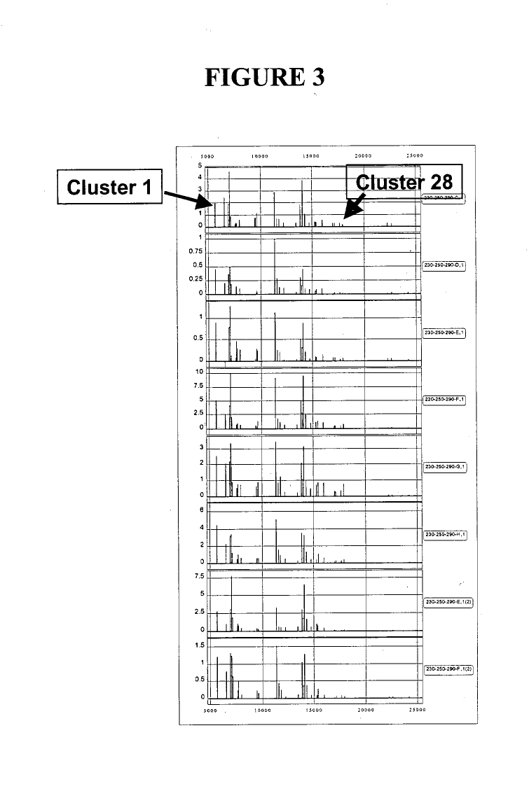

FIG. 4 shows a scatter plot for visualization of pattern differences between subtype I and II of cancer A.

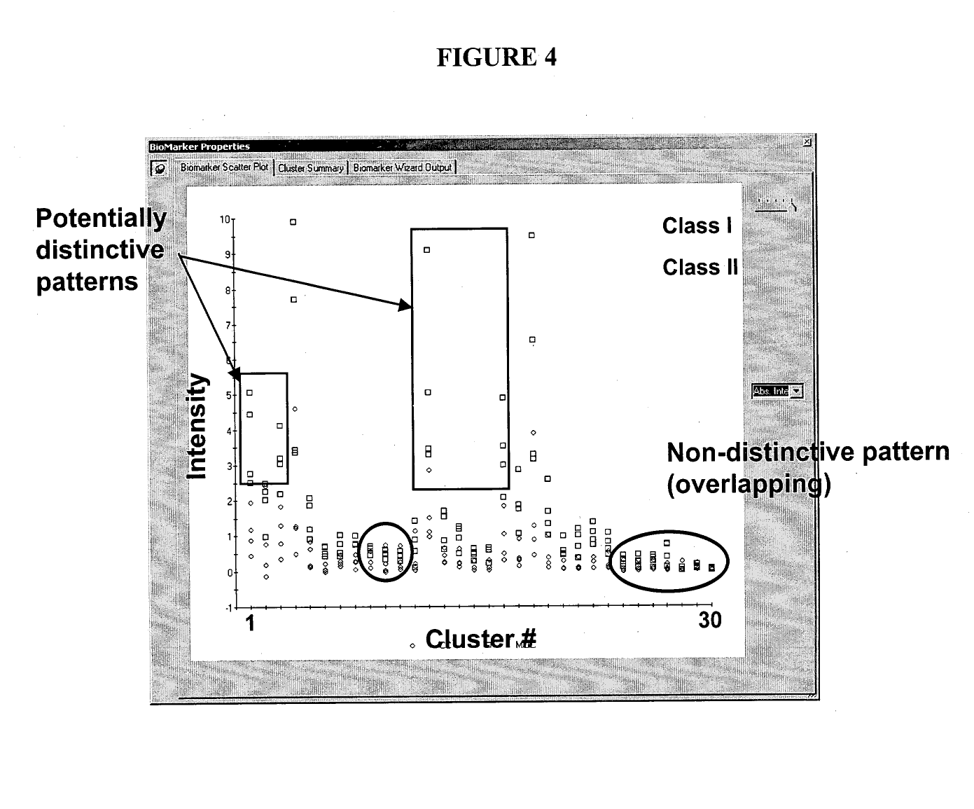

FIG. 5 diagrams of one example of an experimental design for protein profiling in saliva.

FIG. 6 provides spectral and gel views of saliva protein data from a WCX chip, prepared and washed in pH 3.5 buffer.

FIG. 7 provides spectral views of saliva protein data from a WCX chip, prepared and washed in pH 3.5 buffer (left panel), and of sample 5IS on WCX chip prepared and washed at pHs ranging from 3.5 to 9.5.

FIG. 8 provides a graphic representations of the applications for PROTEINCHIP arrays in molecular recognition studies.

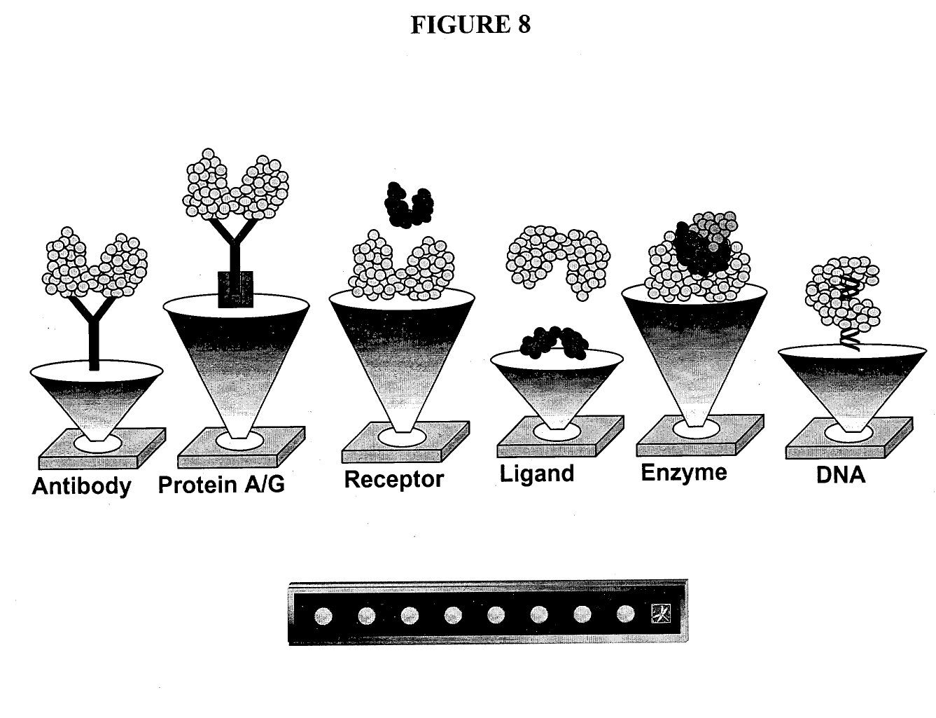

FIG. 9 provides spectral and gel views of saliva protein expression profiling on a WCX chip, prepared and washed in pH 3.5 buffer.

FIG. 10 provides spectral views of saliva protein expression profiling on a WCX chip, prepared and washed in pH 3.5 buffer.

FIG. 11 provides spectral views of saliva protein expression profiling on a WCX chip, prepared and washed in pH 3.5 buffer.

FIG. 12 provides spectral views of saliva protein expression profiling on a WCX chip, prepared and washed at pHs ranging from 3.5 to 7.5

FIG. 13 provides spectral views of saliva protein expression profiling on a WCX chip prepared and washed at pH3.5, showing deglycosylation of a 170 kD protein in a saliva sample.

FIG. 14 provides spectral and gel views of protein expression profiling on a WCX chip prepared and washed at pH3.5, comparing the levels of an approximately 80 kD protein in cell extract, saliva, and serum.

FIG. 15 provides spectral and gel views of protein expression profiling on a WCX chip prepared and washed at pH3.5, comparing the levels of an approximately 100 kD protein in cell extract, saliva, and serum.

FIG. 16 provides spectral and gel views of protein expression profiling on a WCX chip prepared and washed at pH3.5, comparing the peaks at about 100 and 113 kD in cell extract, saliva, and serum.

FIG. 17 provides spectral and gel views of protein expression profiling on a WCX chip prepared and washed at pH3.5, comparing the levels of an approximately 113 kD protein in saliva samples.

FIG. 18 provides spectral and gel views of protein expression profiling on a WCX chip prepared and washed at pH3.5, comparing levels of 110 and 170 kD proteins in cell extract, saliva, and serum.

FIG. 19 provides spectral and gel views of protein expression profiling on a WCX chip prepared and washed at pH3.5, comparing levels of 185, 212, 228 and 287 kD proteins in cell extract, saliva, and serum.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary ofMicrobiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide”, “peptide” and “protein” include glycoproteins and proteins comprising any other modification, as well as non-glycoproteins and proteins that are otherwise unmodified.

“Protein profile”, as used herein, refers to the collection of proteins, protein fragments, or peptides present in a sample. The protein profile may comprise the identities (e.g., specific names or amino acid sequence identities of known proteins, or molecular weights or other descriptive information about proteins that have not been further characterized) of the proteins in a collection, without reference to quantity present. In other embodiments, a protein profile includes quantitative information for the proteins represented in a sample.

“Quantitation”, as used herein in reference to proteins in a profile refers to the determination of the amount of a particular protein or peptide present in a sample. Quantitation can be either in absolute amount (e.g., μg/ml) or a relative amount (e.g., relative intensity of signals).

“Marker” and “Biomarker” are used interchangeably to refer to a polypeptide (of a particular apparent molecular weight) that is differentially present in a samples taken from two different subjects, e.g., from a test subject or patient having a particular medical condition, such as cancer, compared to a comparable sample taken from a control subject (e.g., a person with a negative diagnosis or undetectable cancer; a normal or healthy subject).

The phrase “differentially present” refers to differences in the quantity, frequency or modification of a marker present in a sample taken from a test subject as compared to a control subject. For example, a marker can be a polypeptide that is present at an elevated level or at a decreased level in samples of breast cancer patients compared to samples from control subjects. Alternatively, a marker can be a polypeptide that is detected at a higher frequency or at a lower frequency in samples of breast cancer patients compared to samples of control subjects. In addition, a marker can be a polypeptide that is processed differently (e.g., in post translational cleavage; having greater, lesser or different glycosylation and/or phosphorylation; having different folding). A marker can be differentially present in terms of any or all of quantity, frequency or processing.

“Treatment” as used herein, refers to any medical intervention or therapy given to or performed on a test subject or patient in response to a particular medical condition, e.g., drug therapy, surgery, dietary change, etc.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide one or more subjects' samples is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in an assay, are termed “true negatives.” The “specificity” of a diagnostic assay is 1 minus the false positive rate, where the “false positive” rate is defined as—the proportion of those without the disease who test positive. While a particular diagnostic method may not provide a definitive diagnosis of a condition, it suffices if the method provides a positive indication that aids in diagnosis.

As used herein, a “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's-sample that is consistent with a diagnosis of breast cancer. A diagnostic amount can be either in absolute amount (e.g., μg/ml) or a relative amount (e.g., relative intensity of signals).

A “control amount” of a marker can be any amount or a range of amount that is to be compared against a test amount of a marker. For example, a control amount of a marker can be the amount of a marker in a person without breast cancer. A control amount can be either in absolute amount (e.g., μg/ml) or a relative amount (e.g., relative intensity of signals).

The term “probe” as used herein refers to a device that is removably insertable into a gas phase ion spectrometer and comprises a substrate having a surface for presenting a marker for 15 detection. A probe can comprise a single substrate or a plurality of substrates. Terms such as PROTEINCHIP, PROTEINCHIP array, or chip are also used herein to refer to specific kinds of probes.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.” “Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve”, “resolution”, or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Gas phase ion spectrometer” refers to an apparatus that measures a parameter that can be translated into mass-to-charge ratios of ions formed when a sample is volatilized and ionized. Generally ions of interest bear a single charge, and noise-to-charge ratios are often simply referred to as mass. Gas phase ion spectrometers include, for example, mass spectrometers, ion mobility spectrometers, and total ion current measuring devices.

“Laser desorption mass spectrometer” refers to a mass spectrometer which uses laser as means to desorb, volatilize, and ionize an analyte.

“Detect” refers to identifying the presence, absence or amount of the object to be detected.

“Antibody” refers to a polypeptide ligand substantially encoded by an inummoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad inimunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes 15 polyclonal antibodies, monoclonal antibodies, chimeric antibodies, humanized antibodies, or single chain antibodies. “Fe” portion of an antibody refers to that portion of an immunoglobulin heavy chain that comprises one or more heavy chain constant region domains, CHI, CH2 and CH3, but does not include the heavy chain variable region.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay fort-nats and conditions that can be used to determine specific immunoreactivity). Typically a specific or selective reaction will be at least twice background signal or noise and more typically more than 10 to 100 times background.

“Energy absorbing molecule” or “EAM” refers to a molecule that absorbs energy from an ionization source in a mass spectrometer thereby aiding desorption of an analyte, such as a marker, from a probe surface. Depending on the size and nature of the analyte, the energy absorbing molecule can be optionally used. Energy absorbing molecules used in MALDI are frequently referred to as “matrix”. Cinnamic acid derivatives, sinapinic acid (“SPA”), cyano hydroxy cinnamic acid (“CHCA”) and dihydroxybenzoic acid are frequently used as energy absorbing molecules in laser desorption of bioorganic molecules.

### DETAILED DESCRIPTION OF THE INVENTION

One aspect of the present invention is the creation of profiles of the proteins present in saliva or other fluids (e.g., urine, blood, breast milk, lacrymal fluid, etc.). The methods of the present invention are not limited to any particular method of protein profiling. By way of example, and not intending to limit the methods of the present invention to any particular analytical method, various approaches using mass spectrometry for such profiling are provided below. It is not intended that the methods of the present invention be limited to mass spectrometry, or to any particular method of mass spectrometry.

In some embodiments, saliva-based protein profiles are generated using of electrospray ionization (ESI) and matrix-assisted laser desorption/ionization (MALDI) techniques. In a technique known as peptide mass fingerprinting, mass spectrometry is used to identify proteins purified from biological samples. Identification is effected by matching the mass spectrum of proteolytic fragments of the purified protein with masses predicted from primary sequences prior-accessioned into a database. Roepstorff, The Analyst 117:299-303 (1992); Pappin et al., Curr. Biol. (3.6):327-332 (1993); Mann et al., Biol. Mass Spectrom. 22.338-345 (1993); Yates et al., Anal. Blochem. 213.397-408 (1993); Henzel et al., Proc. Natl. Acad. Sci. USA 90:5011-5015 (1993); James et al., Biochem. Biophys. Res. Commun. 195:58-64 (1993). Similar database-mining approaches have been developed that use fragment mass spectra obtained from collision induced dissociation (CID) or MALDI post-source decay (PSD) to identify purified proteins. Eng et al., J. Am. Soc. Mass. Spectrom. 5:976-989 (1994)); Griffin et al., Rapid Commun. Mass Spectrom. 9:15461551 (1995); Yates et al., U.S. Pat. Nos. 5,538,897 and 6,017,693; Mann et al., Anal. Chem. 66:4390-4399 (1994).

In some embodiments, saliva-based protein profiles are generated using mass spectrometric techniques that permit at least partial de novo sequencing of isolated proteins. Chait et al., Science 262:89-92 (1993); Keough et al., P-roc. Natl. Acad. Sci. USA. 96:7131-6 (1999); reviewed in Bergman, EXS 88:133-44 (2000). Software resources that facilitate interpretation of protein mass spectra and mining of public domain sequence databases are now readily accessible on the internet to facilitate protein identification. Among these are Protein Prospector (available at the UCSF web site), PROWL (available at the Rockefeller University web site), and the Mascot Search Engine (Matrix Science Ltd., London, UK, available through their web site).

In preferred embodiments, saliva-based protein profiled are generated using affinity capture laser desorption ionization approaches. These approaches allow proteins to be profiled without prior purification from complex mixtures. Hutchens et al., Rapid Commun. Mass Spectrom. 7: 576-580 (1-993); U.S. Pat. Nos. 5,719,060, 5,894,063, 6,020,208, 6,027,942 and 6,225,047, each incorporated by reference herein. This strategy for MS analysis of macromolecules uses laser desorption ionization probes that have an affinity reagent on at least one surface. The affinity reagent adsorbs desired analytes from heterogeneous samples, concentrating them on the probe surface in a form suitable for subsequent laser desorption ionization. The coupling of adsorption and desorption of the analyte obviates off-line purification approaches, permitting analysis of smaller initial samples and further facilitating sample modification approaches directly on the probe surface prior to mass spectrometric analysis. The affinity capture laser desorption ionization approach has allowed mass spectrometry to be adapted to numerous classic bioanalytical assay formats, including immunoassay, Nelson et al., Anal. Chem. 67: 1153-1158 (1995) and affinity chromatography, Brockman et al., Anal. Chem. 67. 4581.4585 (1995). The affinity capture laser desorption ionization approach has been applied not only to the study of peptides and proteins, Hutchens et al., Rapid Commun. Mass Spectrom. 7:576-580 (1993); Mouradian et al., J. Amer. Chem. Soc. 118: 8639-8645 (1996); Nelson et al., Rapid Commun. Mass. Spectrom. 9. 1380.1385 (1995); Nelson et al, J. Molec. Recognition 12: 77-93 (1999).; Brockman et al., J, Mass Spectrom. 33. 1141-1147 (1998); Yip et al., J. Biol. Chem. 271. 32825.33 (1996), but also to oligonucleotides, Jurinke et al., Anal. Chem. 69:904-910 (1997); Tang et al., Nucl. Acids Res. 23: 3126-3131 (1995); Liu et al., Anal. Chem. 67: 3482-90 (1995); Bundy et al., Anal. Chem. 71: 1460-1463 (1999), and small molecules, Wei et al., Nature 399:243-246 (1999). An apparatus and methods for efficient affinity capture laser desorption tandem mass spectrometric analysis have been described in WO0223200 to Yip, et al., filed Sept. 7, 2001, incorporated herein by reference in its entirety for all purposes.

At the commercial level, affinity capture laser desorption ionization is embodied in Ciphergen's PROTEINCHIP Systems (Ciphergen Biosystems, Inc. Fremont, Calif. USA)(Davies, et al., Biotechniques 27(6):1258-1261 (1999); von Eggeling, et al., Electrophoresis 2001 22:2898-2909(2001), each incorporated herein in its entirety for all purposes).

The Ciphergen PROTEINCHIP System (series PBS II) includes a PROTEINCHIP Reader integrated with PROTEINCHIP Software and a PC to analyze proteins captured on Ciphergen's PROTEINCHIP Arrays. The PROTEINCHIP System detects proteins ranging from small peptides of less than 1000 Da up to proteins of 300 kilodaltons or more and calculates the mass based on time-of-flight. The PROTEINCHIP software features automatic peak detection; multiple spectrum comparison; several alternative data view formats; and automated chip-reading protocols.

The invention is not limited to analysis using any particular PROTEINCHIP. In some embodiments, saliva proteins are profiled using cation exchange PROTEINCHIP arrays. Cationic arrays bind proteins through electrostatic interaction of positively charged amino acids such as lysine, arginine and histidine. Binding occurs at low pH with low salt; binding decreases as pH and salt concentration increase. In some embodiments, a weak cation exchange array with a carboxylate surface, such as the WCX2 PROTEINCHIP, is used to bind cationic proteins. The negatively charged carboxylate groups on the surface of the WCX2 chip interact with the positive charges exposed on the target proteins. The binding of the target proteins is reduced by increasing the concentration of salt or by increasing the pH of the washing buffers.

In some embodiments, saliva proteins are profiled using anion exchange PROTEINCHIP arrays. Anionic arrays bind proteins through electrostatic interaction of negatively charged amino acids such as aspartic acid and glutamic acid. Binding occurs at high pH with low salt and binding decreases as pH decreases and salt concentration increases. In some embodiments, a strong anion exchange array with a higher-capacity quartenary ammonium surface, such as the SAX2 PROTEINCHIP, is used to bind anionic proteins.

In some embodiments, saliva proteins are profiled using hydrophobic PROTEINCHIP Arrays. Hydrophobic arrays bind proteins through hydrophobic surface interaction with amino acids such as alanine, valine, leucine, isoleucine, phenylalanine, tryptophan and tyrosine. Binding occurs in aqueous, high salt conditions and binding is reduced by decreasing salt and increasing the concentration of organics. In some embodiments, a hydrophobic array containing a long-chain aliphatic surface that binds proteins by reverse phase interaction, such as the H4 PROTEINCHIP is used.

In some embodiments, saliva proteins are profiled using hydrophilic PROTEINCHIP arrays. Hydrophilic arrays bind proteins through electrostatic and dipole-dipole interactions as well as hydrogen binding. Proteins with hydrophilic and charged surface animo acids such as serine, threonine and lysine bind well. Binding occurs in aqueous buffers with a water wash prior to analysis. In some embodiments, hydrophilic arrays contain a SiO2 surface, such as PROTEINCHIPs NP1 and NP2, are used for general binding of proteins.

In yet other embodiments, saliva proteins are profiled using immobilized metal affinity PROTEINCHIP arrays. Immobilized metal affinity capture (IMAC) arrays bind proteins and peptides that have affinity for metals. Proteins with exposed histidine, tryptophan and/or cysteine typically bind to metals immobilized on these chip surfaces. Binding occurs under pH 6-8 and high salt and decreases as the concentration of imidizole and glycine increase. In some embodiments, a PROTEINCHIP array containing a nitriloacetic acid (NTA) surface, such as the IMAC3 PROTEINCHIP array, is used for high-capacity nickel binding and subsequent affinity capture of proteins with metal binding residues. Imidazole may be used in binding and washing solutions to moderate protein binding, including binding of non-specific proteins. Increasing the concentration of imidazole in the washing buffers reduces the binding of the target proteins.

In still other embodiments, saliva proteins are profiled using preactivated PROTEINCHIP arrays. In some embodiments, a preactivated PROTEINCHIP array containing a carbonyldiimidazole surface that covalently reacts with amine groups, such as the PS1 PROTEINCHIP array, is used. DNA and proteins, including antibodies, can be immobilized on the PS1 surface. In other embodiments, a preactivated PROTEINCHIP array containing an epoxy surface which covalently reacts with amine and thiol groups, such as the PS2 PROTEINCHIP array, is used. DNA and proteins, including antibodies, can be immobilized on the PS2 surface.

Salivary markers have been described for several cancers. For example, Chien found that saliva contained CA 125, a glycoprotein complex that is a recognized or accepted tumor marker for epithelial ovarian cancer. (Chien D X, Schwartz P E, CA 125 Assays for Detecting Malignant Ovarian Tumors. Obstetrics and Gynecology, 75(4):701-704, 1990). In comparing salivary CA 125 concentrations among healthy controls, women with benign lesions, and those with ovarian cancer, Chien found a significantly elevated CA 125 concentration among the ovarian cancer group as compared to the nonmalignant controls. Streckfus, et al., described a method of using a salivary biomarkers to differentially diagnose and/or detect reoccurrence of breast carcinoma (Streckfus C., et al., Clinical Cancer Research 6:2363-2370 (2000); Streckfus C., et al., Oral Surgery, Oral Medicine Oral Pathlogy 91(2):174-179 (2002); U.S. Pat. No. 6,294,349; and international patent application WO 00/52463, each incorporated by reference herein in their entirety).

The present invention provides rapid and cost-effective methods for identifying biomarkers for additional phenotypes, conditions, diseases and the like.

### Experimental Examples

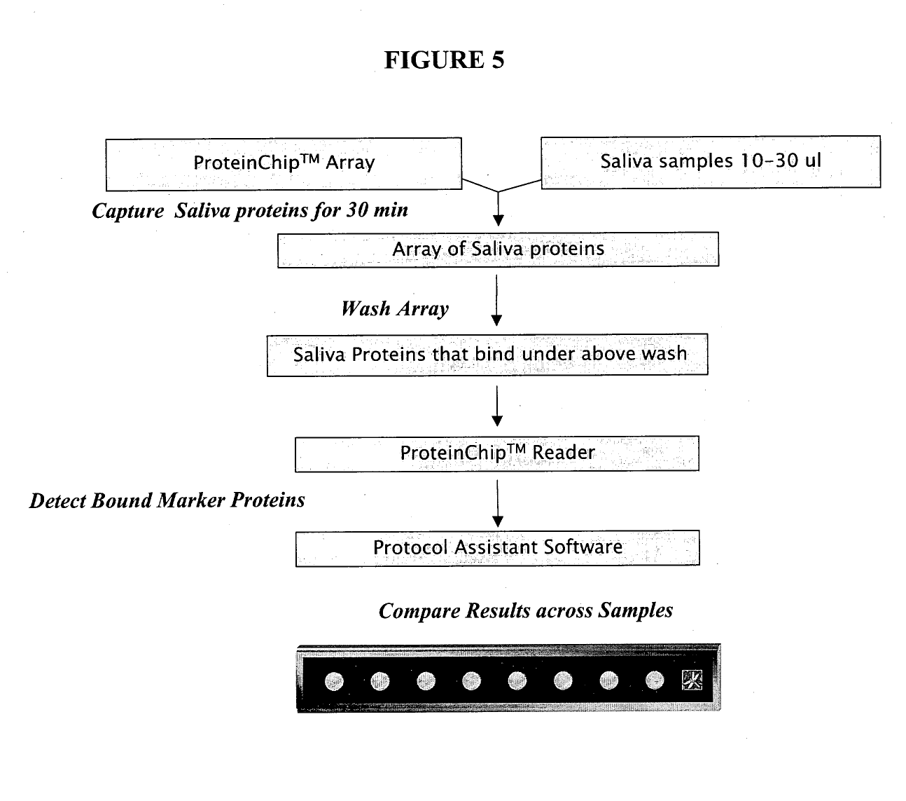

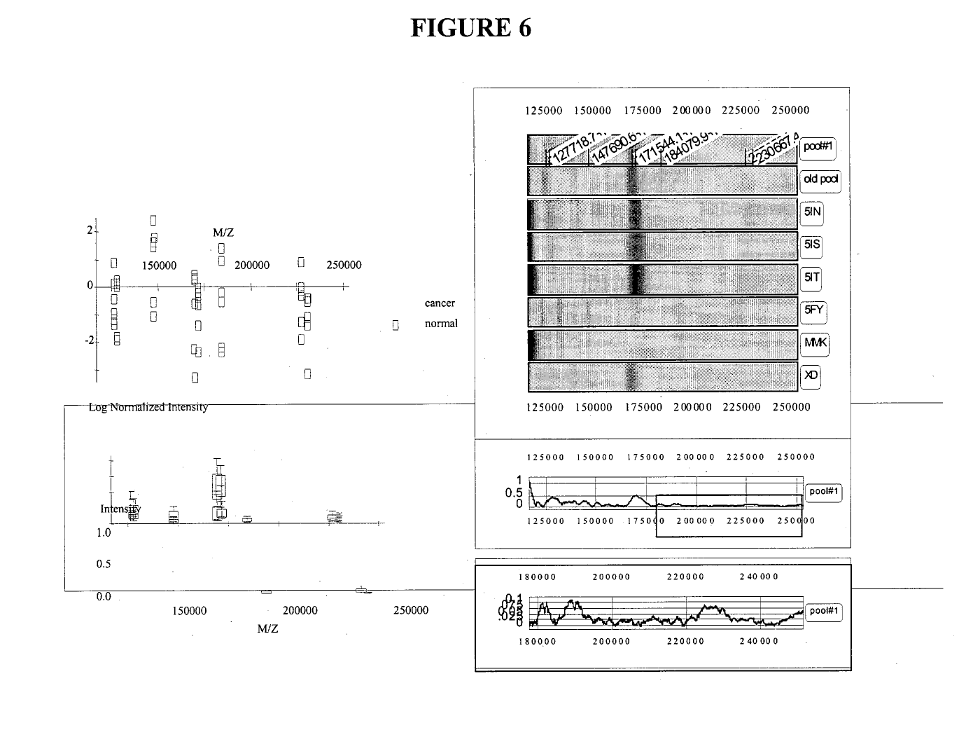

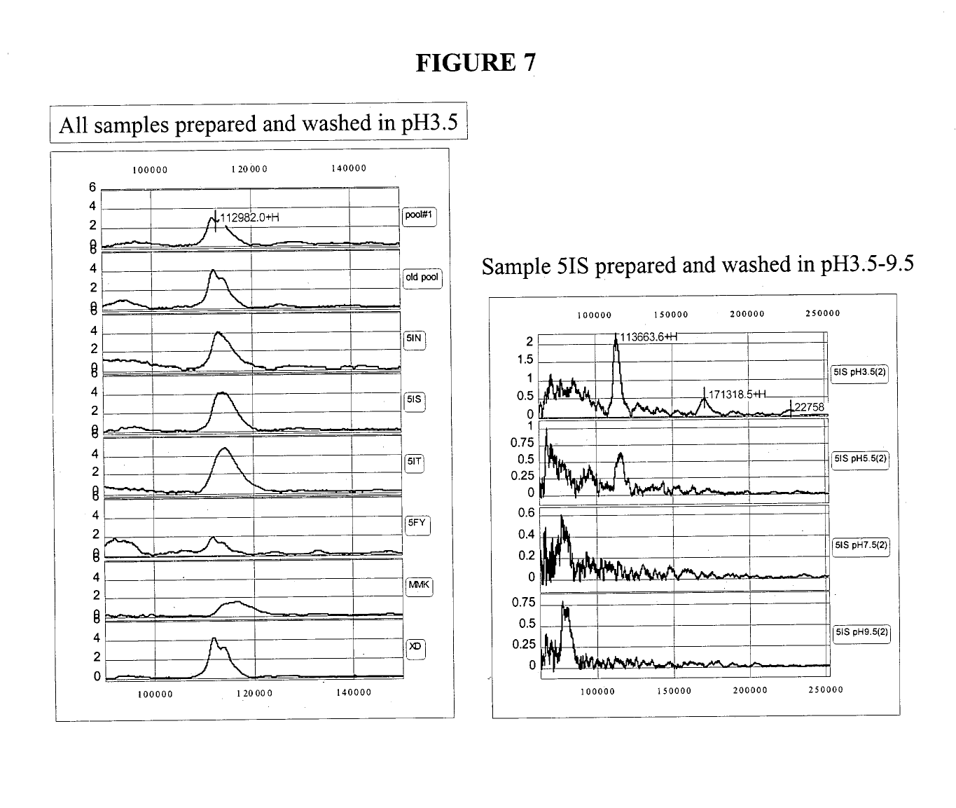

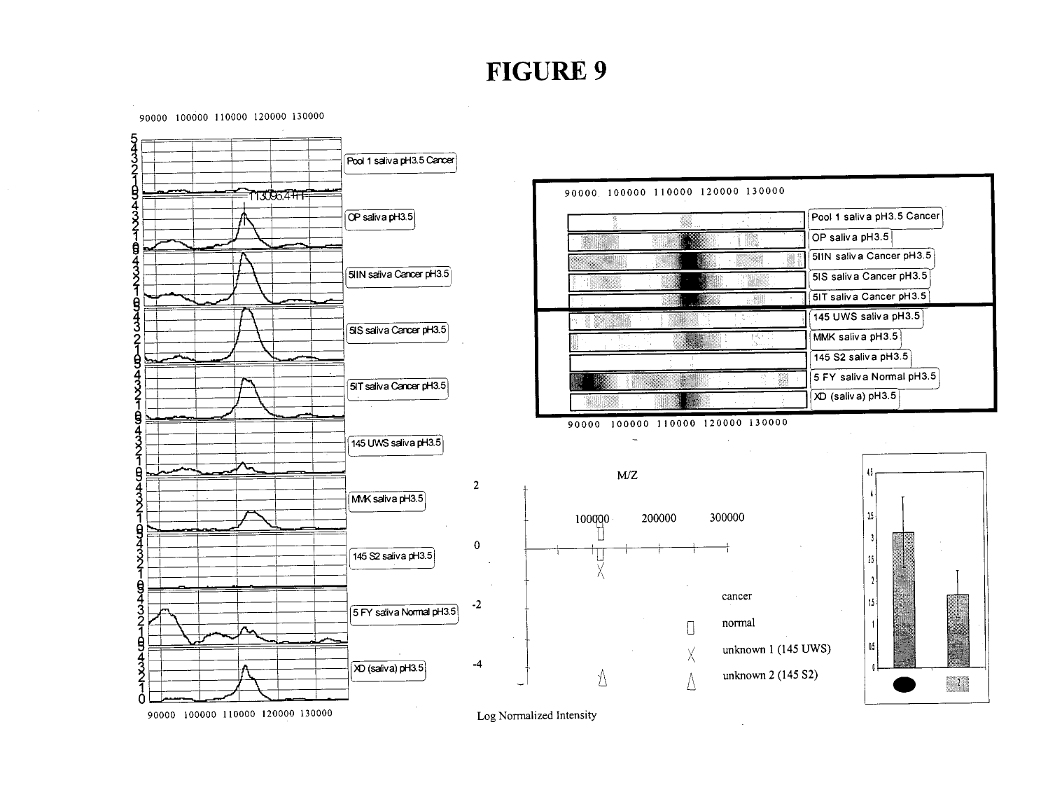

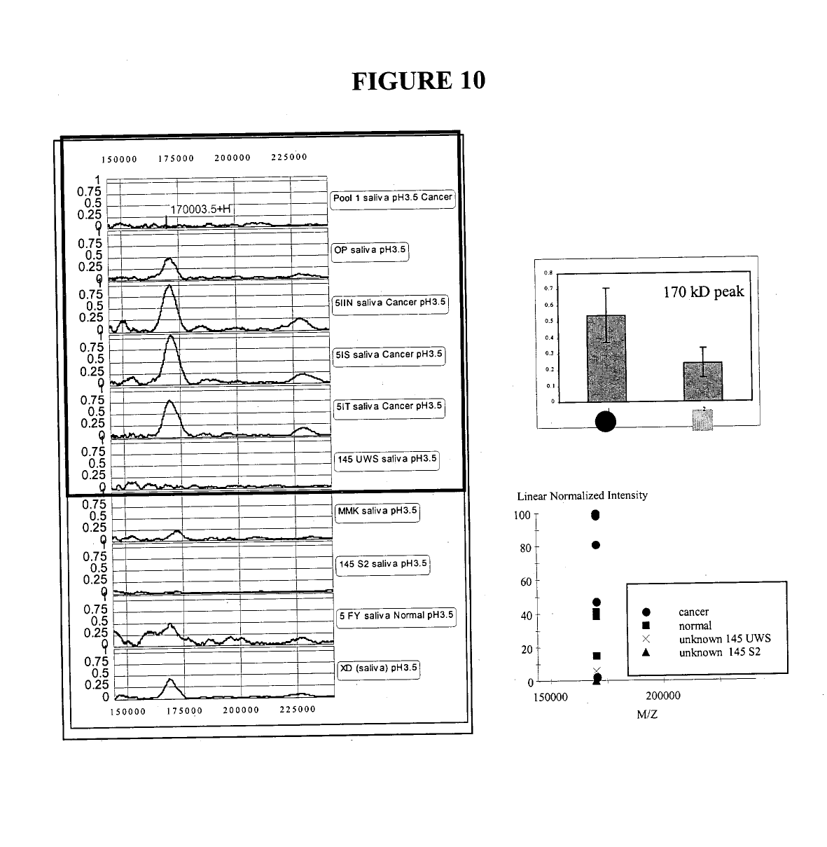

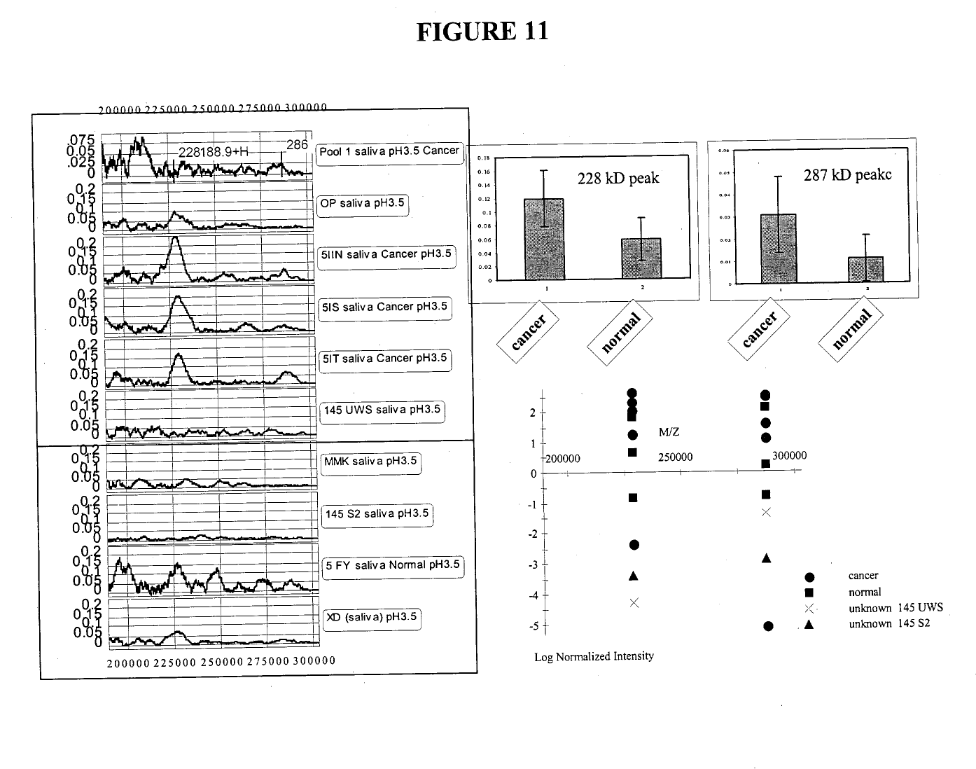

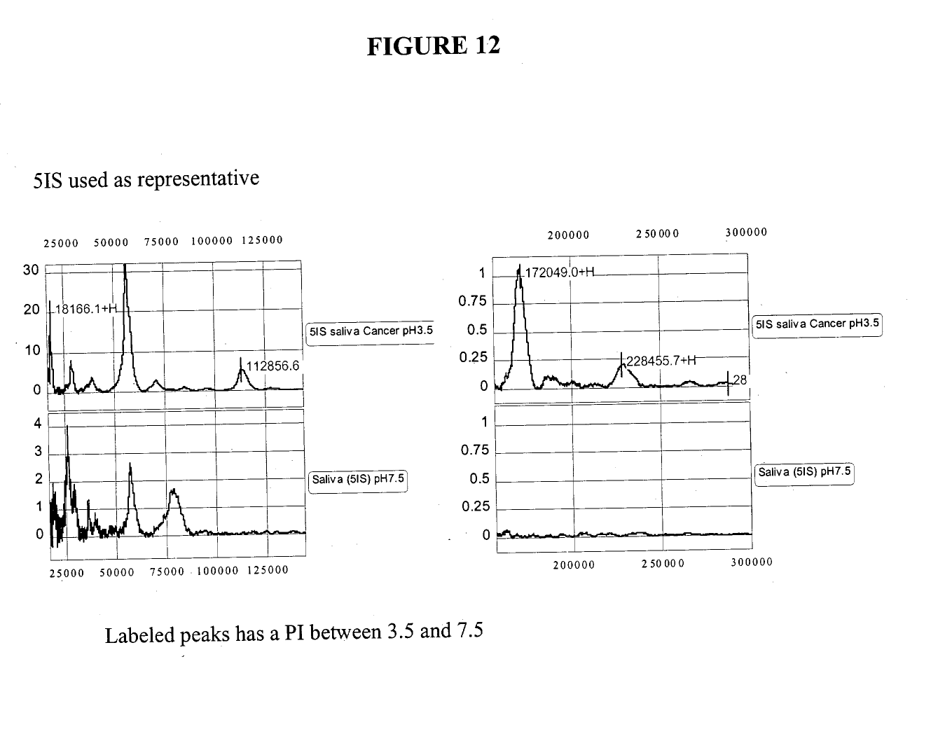

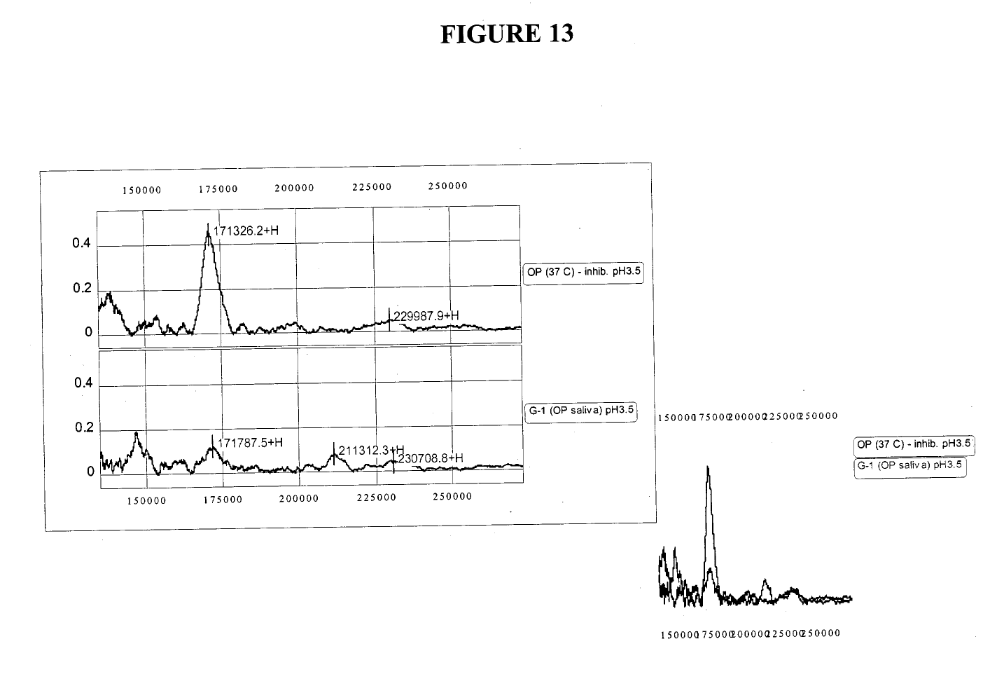

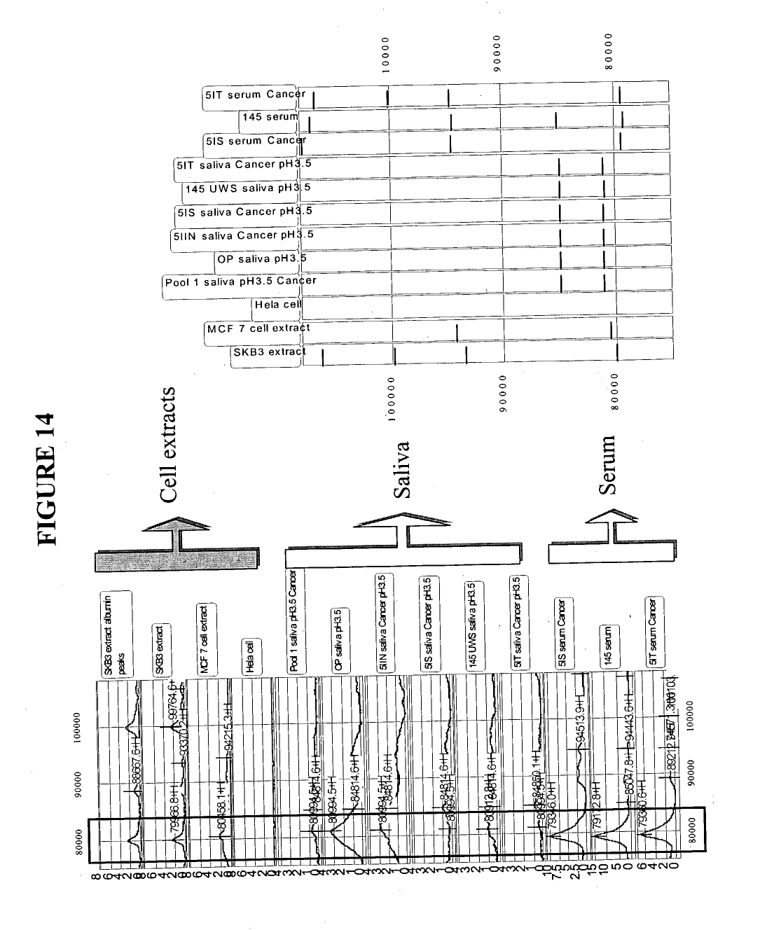

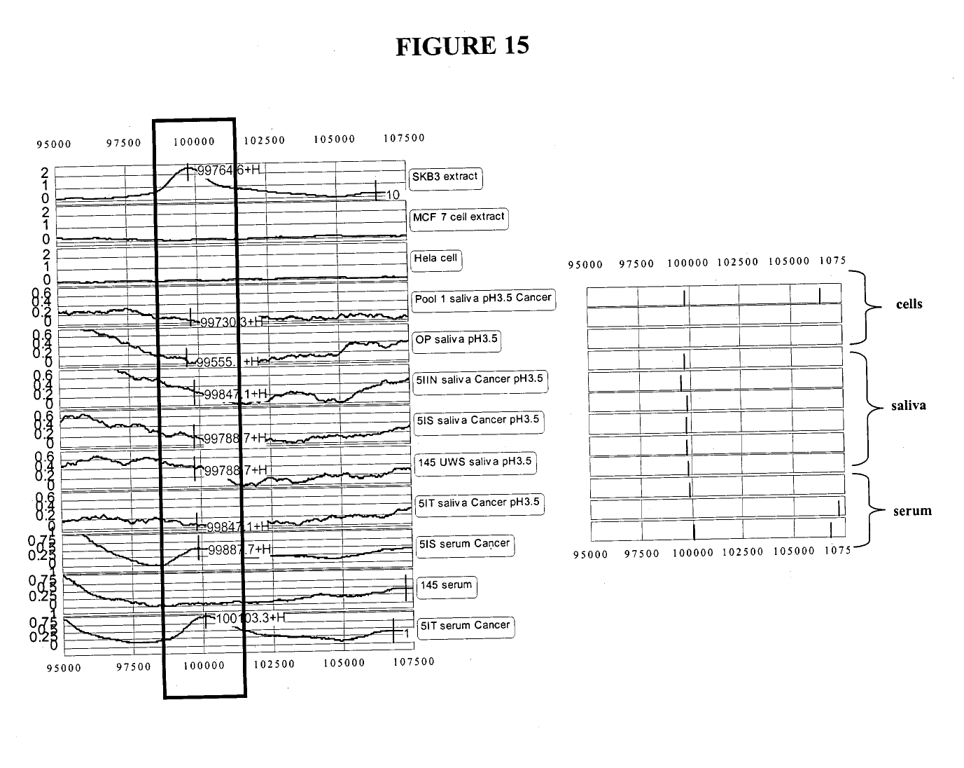

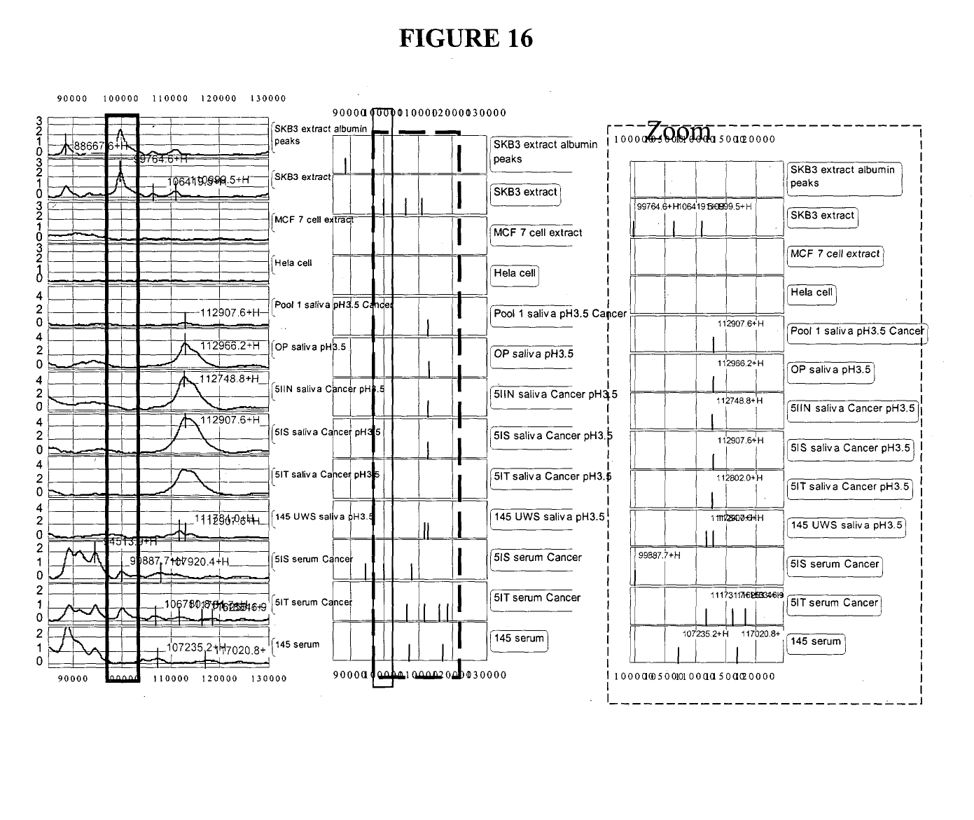

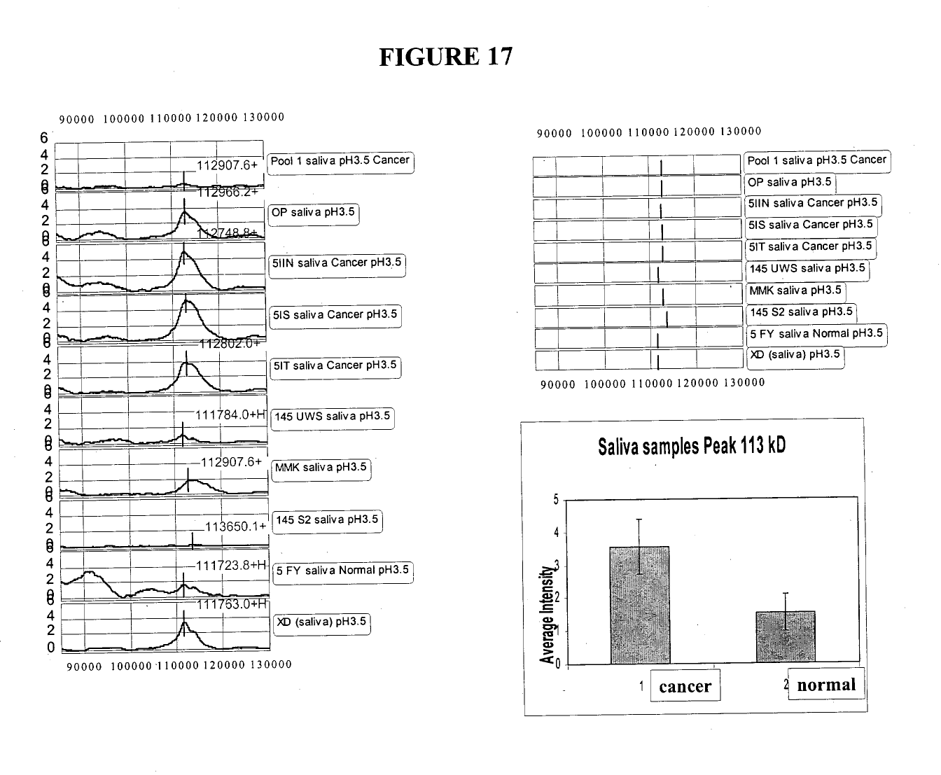

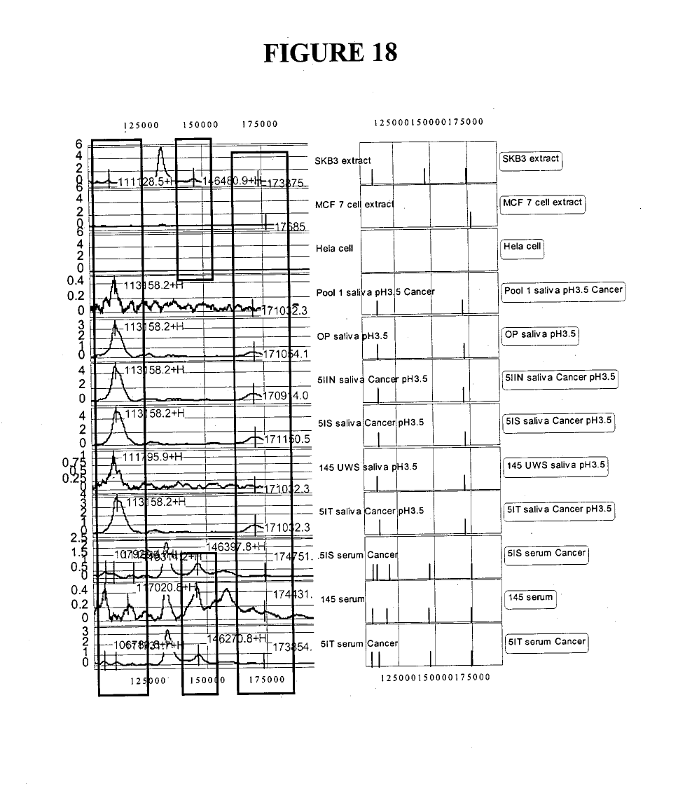

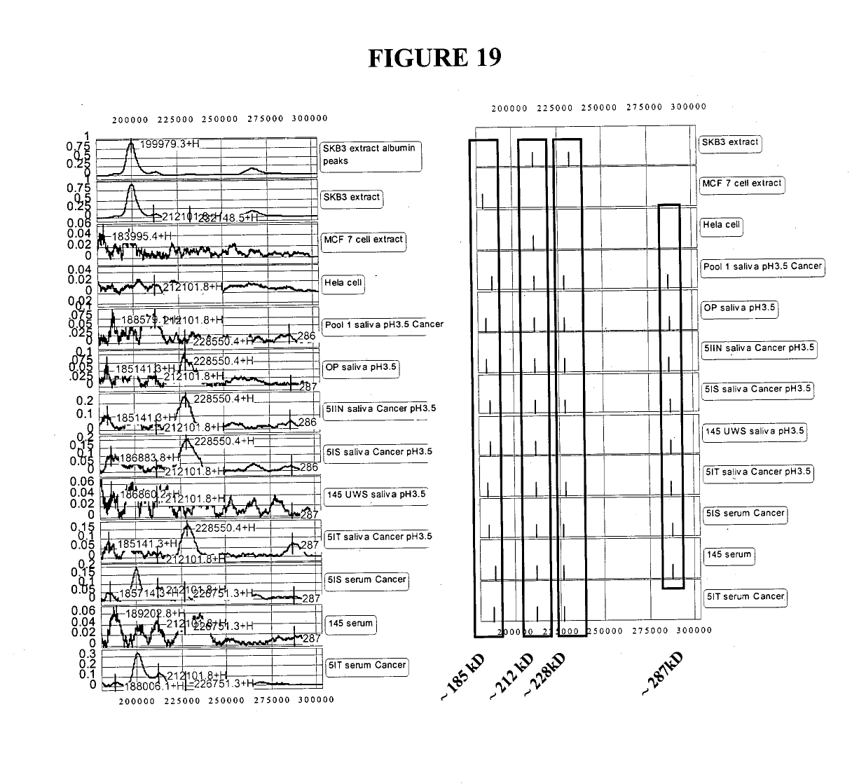

FIG. 5 provides diagrams one example of an experimental design for protein profiling in saliva. FIGS. 6, 7, and 9-19 provide examples of protein profiles generated from saliva and other fluids, as indicated, under the variety of conditions indicated. For the WCX PROTEINCHIP arrays (Ciphergen, Inc.), 10 μl of saliva was diluted into 140 μl of 100 mM sodium acetate buffer, pH 3.5. Samples were applied to the chip, washed, dried and detected according to manufacturers instructions. The profiles are shown in spectral and gel display formats in these figures.

All publications and patents mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims

What is claimed is:

1. A method for detecting proteins that are differentially present in a first and a second saliva sample comprising the steps of:
a) providing a first saliva sample and a second saliva sample,
b) determining a first protein profile for said first saliva sample;
c) determining a second protein profile for said second saliva sample; and
d) comparing said first protein profile and said second protein profile to detect proteins that are differentially present in the first and second saliva sample.

2. The method of claim 1, wherein said first and said second saliva samples are from a test subject and a control subject, respectively.

3. The method of claim 2, wherein said test subject is a person having a particular medical condition and wherein said control subject is a person with a negative diagnosis for said particular medical condition.

4. The method of claim 1, wherein said first and said second saliva samples are collected from a single test subject.

5. The method of claim 4, wherein said first and said second saliva samples are collected at a first time point and a second time point from said test subject.

6. The method of claim 5, wherein said test subject develops a particular medical condition at a third time point between said first time point and said second time point.

7. The method of claim 5, wherein said test subject has a particular medical condition.

8. The method of claim 7, wherein said test subject receives a treatment at a third time point between said first time point and said second time point.

9. The method of claim 1, wherein said first and said second protein profiles are determined by mass spectrometry.

10. The method of claim 9, wherein said mass spectrometry comprises affinity capture laser desorption ionization.

11. The method of claim 1, wherein said first and said second protein profiles are determined using protein chip array analysis.

12. The method of claim 11, wherein said protein chip array analysis comprises SELDI PROTEINCHIP array analysis.

13. A method of identifying a biomarker in saliva for a particular phenotype, comprising:
a) providing a first saliva sample from a test subject having a phenotype;
b) providing a second saliva sample from a control subject not having said phenotype,
c) determining a first protein profile for said first saliva sample;
d) determining a second protein profile for said second saliva sample; and
e) comparing said first protein profile and said second protein profile to detect proteins that are differentially present in the first and second saliva sample.
